(19) United States
(12) Patent Application Publication
(10) Pub. No.: US 2014/0348772 A1
(43) Pub. Date: Nov. 27, 2014

(54) PRODUCTION OF HYDROGELS BY MEANS OF DIELS-ALDER REACTION

(71) Applicant: Universitaet Regensburg, Regensburg (DE)

(72) Inventors: Achim Goepferich, Sinzing (DE); Ferdinand Brandl, Regensburg (DE); Susanne Kirchhof, Regensburg (DE)

(21) Appl. No.: 14/271,827

(22) PCT Filed: Nov. 7, 2012
(86) PCT No.: PCT/EP2012/072027

§ 371 Date: May 7, 2014

(30) Foreign Application Priority Data
Nov. 8, 2011 (DE) 10 2011 117 839.6

(51) Int. Cl. A61K 47/48; A61K 31/765; A61K 31/787

(52) U.S. Cl. CPC A61K 47/48215; A61K 31/787; A61K 31/765
USPC 424/78.3; 514/772.1; 424/93.1

(57) ABSTRACT

The present invention is directed to hydrogels and their preparation by cross-linking macromonomers by means of Diels-Alder reaction. The hydrogels described in the invention, which can gel in situ, are suitable, inter alia, as biomaterial for medical applications, as scaffolding material for living cells and as carrier system for the controlled release of drugs.

### FIELD OF THE INVENTION

The present invention is directed to hydrogels and their preparation by cross-linking macromonomers by means of Diels-Alder reaction. The hydrogels described in the invention, which can gel in situ, are suitable, inter alia, as biomaterial for medical applications, as scaffolding material for living cells, and as carrier system for the controlled release of drugs. For preparing the hydrogels, macromolecules are functionalized with dienes and dienophiles; the number of functional groups (diene or dienophile) is at least two per macromonomer. In the [4+2]-cyclo addition taking place afterwards, the described macromonomers react in an aqueous solution within or outside of the animal or human organism to form covalently cross-linked hydrogels. By modifying the macromonomers with short peptides (e.g. enzymatically cleavable sequences and/or peptides promoting cell adhesion) the hydrogels can be adapted to the requirements of different applications. The Diels-Alder reaction used for cross-linking furthermore allows the functionalization of the hydrogels with further molecular components for the detection, marking and active interaction with their environment.

Drugs of biogenic origin, such as e.g. peptides, proteins or nucleic acids, are becoming increasingly important in numerous innovative therapeutic approaches. In order to be able to unfold their full therapeutic potential, most peptides, proteins or nucleic acids have to be “packaged” in suitable carrier systems. Hydrogels offer ideal parameters to satisfy the strict requirements for carrier systems for drugs of biogenic origin. Hydrogels represent three-dimensional networks obtained by cross-linking natural or synthetic polymers. They are able to absorb and bind many times their dry weight in water. Due to their excellent biocompatibility and their high permeability for nutrients and metabolites, hydrogels are already being used in numerous biomedical applications. Especially interesting for biomedical applications are in situ gelling hydrogels which can be injected as liquid polymer solutions and then solidify into a gel at the application site. The application by means of minimally invasive methods is considered to be especially gentle for the patient. It is the goal of this strategy to release the peptides, proteins or nucleic acids from the hydrogel after gelling for them to attain their effect locally or systemically.

### BACKGROUND OF THE INVENTION

Numerous approaches for the preparation of hydrogels have been described in the literature based on polymers of biological (e.g. polysaccharides) or synthetic (e.g. polyacrylates or polyethylene glycol) origin. The cross-linking of the individual polymer chains can be achieved both by chemical (covalent) and physical bonds (e.g. hydrophobic or ionic interaction). In view of their high stability and extensive insensitivity to external influences (e.g. change of the pH value or ionic strength), covalently cross-linked hydrogels are especially suitable for biomedical applications. However, the cross-linking reactions commonly used in polymer chemistry (such as e.g. free radical polymerizations, polycondensation or polyaddition reactions) often rely on toxic coupling agents, catalysts or photoinitiators. Therefore, the prepared hydrogels have to be subjected to complex and thus expensive purification steps in order to remove toxic impurities and/or unpolymerized monomers. Most of the time, the actual desired injection as a polymer solution and subsequent cross-linking at the application site is out of the question. Due to the high number of functional groups (e.g. alcohols, amines, carboxylic acids, and the like), an uncontrolled and therefore activity-decreasing reaction with the peptides, proteins or nucleic acids present can frequently not be avoided. These two disadvantages often preclude an application of chemically cross-linked hydrogels as carrier systems for drugs of biogenic origin.

So-called click reactions offer an interesting alternative to conventional cross-linking reactions. Click reactions are chemical reactions which proceed under simple reaction conditions (e.g. in an aqueous medium) and with utmost efficiency to yield only the desired reaction products. Due to the orthogonality of the groups participating in the click reactions, a reaction with other functional groups (e.g. from biogenic drugs) can largely be prevented. In general, according to the concept of orthogonality, functional groups can be combined freely without any undesired reaction taking place between the groups that are present. However, the well known azide-alkyne cyclo addition (1,3-dipolarcyclo addition, Huisgen reaction) catalyzed by copper (I), which is often carried out in organic chemistry, exhibits similar disadvantages as conventional cross-linking reactions: Due to the high toxicity of the copper (I) catalyst, it is imperative that the resulting hydrogels be subjected to the complex and therefore expensive purification steps. A direct injection into an animal or human organism is out of the question here as well.

The Diels-Alder reaction meets all the criteria of a click reaction (inter alia, easy reaction conditions and high efficiency) and represents an extremely interesting possibility of covalently cross-linking macromonomers. The Diels-Alder reaction is a single-step [4+2]-cyclo addition between a diene and a dienophile. Typically, dienophiles are used which have a C≡C double bond. However, compounds can be reacted as dienophiles in a Diels-Alder reaction which have a double bond between a carbon atom and a heteroatom (e.g. N, O or S) or between two heteroatoms (e.g. N—N or N-0), such as e.g. an aldehyde, a ketone, an imine or a thioketone. Diels-Alder reaction can be schematically represented for example by the following reaction equation (1).

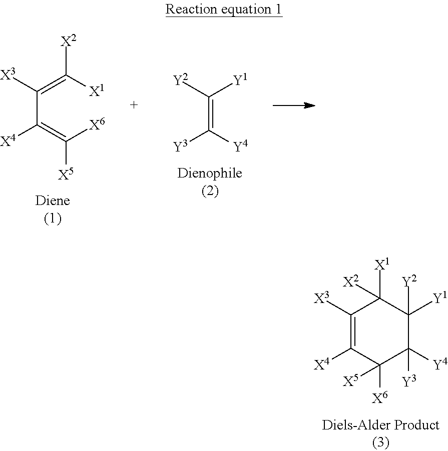

The equation shows a general schematic of Diels-Alder [4+2]-cyclo addition between a diene of the formula (1) and a dienophile of the formula (2), forming a Diels-Alder product of the formula (3). The groups X¹ to X⁶ and Y¹ to Y⁴ represent any desired atoms or molecule groups.

The reaction takes place at an accelerated rate in an aqueous medium; the addition of catalysts or initiators is completely unnecessary. By suitably selecting the substituents X¹ to X⁶ and Y¹ to Y⁴, the rate of the reaction can be influenced. In order to accelerate the reaction, electron-donating substituents (groups with +I and/or +M-effect, e.g. alkyl groups) are selected for X¹ to X⁶; electron-withdrawing substituents (groups with −I and/or −M-effect) are preferably used for Y¹ to Y⁴. For example, dienes of the formula (1) can independently carry hydrogen atoms, hydrocarbon groups (such as e.g. alkyl groups), or alkoxy groups (in particular methoxy groups) as groups X¹ to X⁶. Furthermore, in particular the groups X¹ and X⁶ can be linked to each other, thus forming a carbocyclic or heterocyclic five- or six-membered ring system, such as a furan ring, a cyclopentadienering or a 1,3-cyclohexadiene ring.

Dienophiles of the formula (2) can for example carry a hydrogen atom or a hydrocarbon group, e.g. an alkyl group, at two or three of the positions Y¹ to Y¹ and at the remaining position(s) an electron-withdrawing group such as an aldehyde function —C(O)H, a keto function —C(O)R, an ester function —C(O)OR, a cyano group or a nitro group, wherein R is a hydrocarbon group, e.g. an alkyl group. Also well suited as dienophiles are compounds of the formula (2) wherein Y² and Y³ represent a hydrogen atom or a hydrocarbon group, e.g. an alkyl group, and Y¹ and Y⁴ are linked to each other by forming a maleic acid anhydride, a maleimide or a 1,4-quinone.

As is common for addition reactions, the Diels-Alder reaction does not lead to the release of low-molecular and potentially toxic byproducts. Furthermore, undesired side reactions with other functional groups can largely be excluded. This is due to the orthogonality of the functional groups used in the cross-linking reaction (diene and dienophile). Under the selected conditions, these functional groups react exclusively with each other and can be combined as desired with other functional groups (e.g. alcohols, amines, carboxylic acids, etc.); the use of protecting groups is not necessary. This is of special value for the intended use of the hydrogels of the present invention since uncontrolled and therefore activity-decreasing reactions with the peptides, proteins or nucleic acids present are largely eliminated. In addition to the advantages already mentioned, the macromonomers used in the present invention (diene and dienophile) also show excellent storage stability; this is how they differ from other frequently used derivatives (e.g. activated carboxylic acids or thiols).

Not least because of the advantages described above, the Diels-Alder reaction has been used in polymer chemistry before. There are examples in the literature where Diels-Alder reaction was used in the preparation of elastomers. Elastomers are elastically deformable plastic materials whose glass transition temperature is below their operating temperature. A significant difference to the hydrogels of the present invention is their extremely low water content. Their possible fields of application therefore differ considerably from the fields of application suggested in the present invention. There are also examples in the literature where Diels-Alder reaction was used to prepare hydrogels. For example, high-molecular polyacryl amides, polyacrylates and polyvinylpyrrolidones were cross-linked by means of Diels-Alder reaction. This kind of cross-linking typically results in hydrogels with a high sol fraction and a low-definition architecture; mesh width, swelling degree and mechanical properties are comparatively hard to control. Additional disadvantages of the existing hydrogels are the use of polymers which have not been approved and their non-existent biodegradability. Each of these points usually prevents the use of the hydrogels in biomedical fields. Furthermore, the hydrogels described in the literature are often not optically transparent. This precludes, for example, their use in ophthalmological applications. In a different approach, hyaluronic acid was cross-linked by means of Diels-Alder reaction. The critical disadvantage in this connection is the use of polymers of biological origin. This means that a high degree of variability of the starting material, preparation processes which are hard to reproduce, high production costs and potentially allergenic resulting hydrogels can be expected. Furthermore, it is difficult to chemically alter the hyaluronic acid used in the process. The adjustment of the hydrogels to the requirements of a certain application (e.g. degradability, cell adhesion, release rate, etc.) is extremely difficult.

### BRIEF DESCRIPTION OF DRAWINGS

FIG. 1 show a schematic representation of the cross-linking of macromonomers by means of Diels-Alder reaction. The example shows star-shaped macromonomers with a degree of branching of four.

FIG. 2 shows a covalently cross-linked hydrogel loaded with biogenic drugs (e.g. peptides, proteins or nucleic acids). The hydrogel serves as carrier system and allows a local or systemic therapy for an extended period of time. The example shows star-shaped macromonomers with a degree of branching of four.

FIG. 3 shows a rheogram of a hydrogel with 10% (w/v) total polymer content. PEG macromonomers (molecular weight 10 kDa, degree of branching four) functionalized with furyl groups and maleimide groups were used for the preparation of the gel. Cross-linking took place in water at 37° C. by means of Diels-Alder reaction.

FIG. 4 shows the strength of the Diels-Alder hydrogels based on the degree of branching and the total polymer concentration. The higher the degree of branching and the higher the total polymer concentration, the stronger the gels that can be formed.

FIG. 5 shows the gelling time of the Diels-Alder hydrogels based on the degree of branching and the total polymer concentration. The higher the degree of branching and the higher the total polymer concentration, the faster gel formation begins.

FIG. 6 shows the swelling and degradation behavior of the Diels-Alder hydrogels. The gels show different stability depending on the degree of branching and the selected release medium.

FIG. 7 shows the swelling and degradation behavior based on the total polymer concentration. The stability of the gel cylinders can be prolonged by increasing the total polymer concentration.

FIG. 8 shows the recovery of fluorescence in the bleached area based on time in a FRAP analysis of 4arm PEG-hydrogels and 8armPEG-hydrogels according to the present invention which are loaded with FD150 as model substance.

FIG. 9 shows the prediction regarding the release behavior of FD150 from 4armPEG-hydrogels and 8armPEG-hydrogels.

### SUMMARY OF THE INVENTION

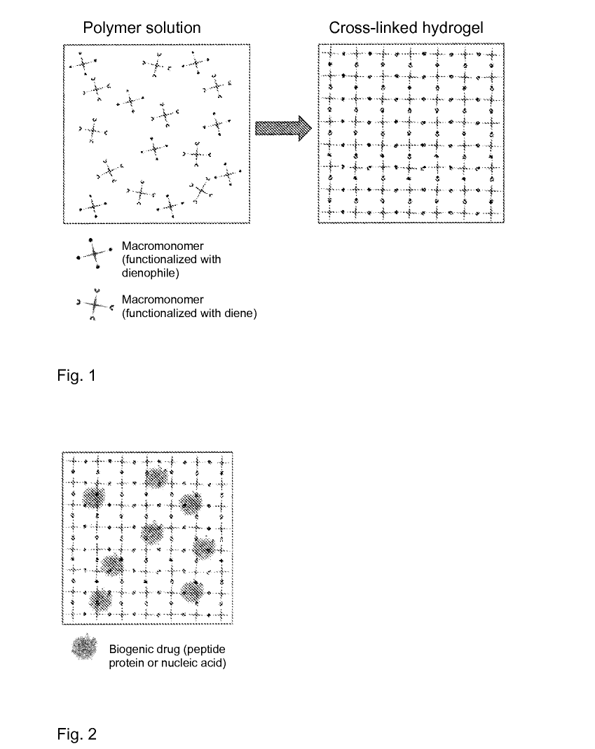

The present invention is therefore directed to a process for the preparation of a hydrogel by cross-linking macromonomers by means of Diels-Alder reaction, as well as hydrogels obtainable by this process. Preferably, the components for the preparation of a hydrogel listed herein are capable of forming the hydrogel in situ, e.g. after application in an organism. FIG. 1 shows a schematic representation of the cross-linking reaction.

Macromonomers which can be used for the preparation of the hydrogels according to the present invention are polymers (also referred to as macromolecules) having functional groups based on which they can react as monomers in a polymerization reaction. Within the framework of the present invention, this reaction is a Diels-Alder reaction between accordingly functionalized macromonomers having diene or dienophile functions.

As starting polymers to provide the macromonomers, hydrophilic polymers are preferably used, in particular hydrophilic polymers of synthetic origin. Hydrophilic polymers of synthetic origin which are approved by the FDA (U.S. Food and Drug Administration) for biomedical applications are especially preferred. As an example of an especially suitable starting polymer is polyethylene glycol (PEG) (also referred to aspolyethylene oxide (PEO)), whereby the reference to polyethylene glycol (PEG) or polyethylene oxide includes branched structures. This water-soluble, biocompatible, non-toxic and nonimmunogenic polymer is already being widely used in biotechnology and medicine. Further examples of macromonomers suitable for use in the present invention include polyvinylalcohol (PVA), polypropylene oxide (PPO), copolymers of ethylene oxide and propylene oxide (PEO-co-PPO), poly(hydroxyethylmethacrylate) (pHEMA), hyaluronic acid, dextran, collagen, chitosan, alginate, cellulose and cellulose derivatives, such as carboxymethylcellulose. The macromonomers used in the present invention are preferably characterized by a relatively low molecular weight (typically 2-20 kDa). Furthermore, a defined architecture is advantageous. Typically, star or comb polymers are used, for example those with a degree of branching of two to eight, preferably three to eight, and especially preferred four to eight. The degree of branching indicates the number of polymer chains extending from the branch point or branch points of the polymer. Accordingly, hydrophilic star or comb polymers of synthetic origin are especially preferred, in particular those with the degree of branching mentioned above.

The following structural formulas show examples of branched PEG molecules suitable for use in the present invention. The number of repeating units n can be adjusted such that the desired molecular weight of the entire molecule is achieved.

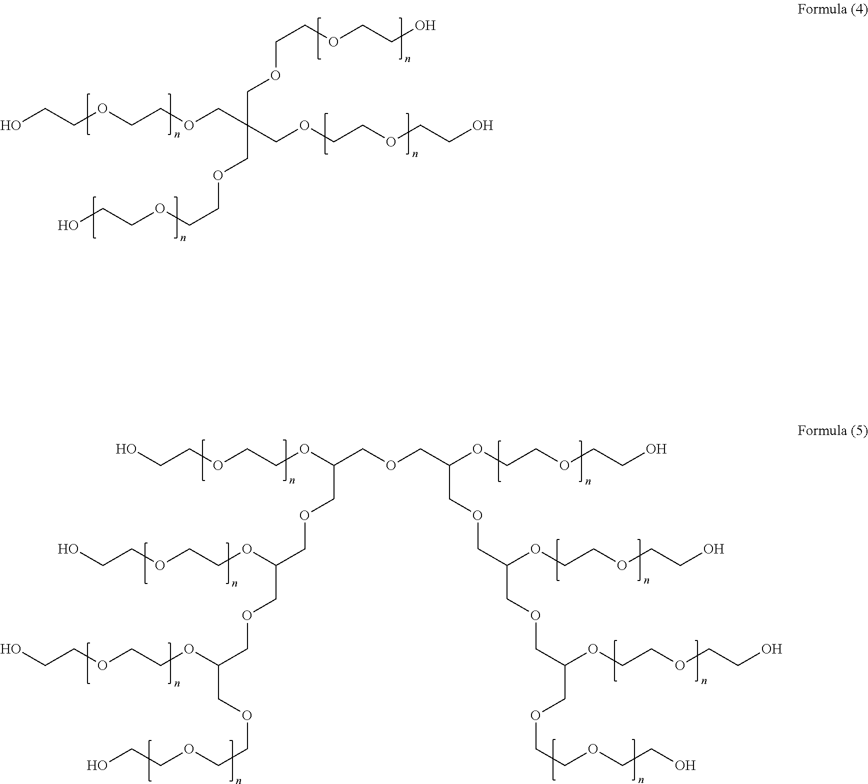

Formula (4) shows a 4-arm branched PEG molecule (degree of branching 4), here also referred to as “4armPEG-OH”. It can be used in different molecular weights, for example with a molecular weight of 10,000 Da (4armPEG10 k-OH).

Formula (5) shows an 8-arm branched PEG molecule (degree of branching 8), here also referred to as “8armPEG-OH”. It can also be used in different molecular weights, for example with a molecular weight of 10,000 Da (8armPEG10 k-OH).

For providing the macromonomers as reactants in a Diels-Alder cross-linking reaction, the selected starting polymers are functionalized with at least two dienes, or at least two dienophiles, respectively. Thus, a macromonomer should preferably show either only diene functions or only dienophile functions since this facilitates a controlled reaction between diene and dienophile components. If macromonomers with mixed diene and dienophile functions are to be used, it would have to be ensured that they do not enter into uncontrolled intra- or inter-molecular reactions during their provision or storage. While both starting polymers are functionalized with at least two diene functions or at least two dienophile functions, the two reactants are usually adjusted to each other such that cross-linking can proceed effectively. For example, if a macromonomer only has two functional groups for Diels-Alder reaction, it will usually be combined with another macromonomer which has three or more functional groups. Preferably, each starting polymer molecule is functionalized with at least three, particularly at least four, dienes or with at least three, particularly at least four, dienophiles in order to form a macromonomer. If star or comb polymers are used, it is especially preferred that each arm of these polymers be functionalized with a terminal diene or dienophile.

Known methods of covalent bonding can be used to introduce a diene or dienophile function, for example by forming an ester or amide bond. With reference to the schematic in reaction equation 1, one or more substituents X¹ to X⁶ or Y¹ to Y⁴ can serve to bond a diene or dienophile to a polymer.

As illustrated above in reaction equation 1, open chain or cyclic compounds which have two conjugated C≡C double bonds are typically used as dienes in Diels-Alder reactions, whereby the double bonds in open chain compounds are present in a cisoid conformation. Examples of such dienes include those of formula (1) above. Dienes with a furan ring, a cyclopentadienering or a 1,3-cyclohexadiene ring are preferred. Dienophiles are typically compounds with a C≡C double bond which are described in formula (2) above. However, compounds which have a double bond between a carbon atom and a heteroatom (e.g. N, O or S) or between two heteroatoms (e.g. N—N or N—O), such as e.g. an aldehyde, aketone, an imine or a thioketone, can also be reacted as dienophiles in a Diels-Alder reaction. Heterocyclic compounds such as 4-phenyl-1,2,4-triazole-3,5-dione (PTAD) can be used as dienophiles as well. Dienophiles comprising a maleic acid anhydride group, a maleimide group or a 1,4-quinone group are preferred. In particular, electron rich furyl groups can be used as dienes; electron poor maleimide groups can be used as dienophiles. Due to this manner of terminal group modification with relatively small groups, the solubility of the derivatized polymer hardly changes so that the macromonomers in which water-soluble polymers are used can still be water-soluble compounds. The macromonomers synthesized with this method can moreover be considered biocompatible.

For preparing the hydrogels, the synthesized macromonomers can be dissolved separately in a defined amount of water or buffer solution (e.g. phosphate-buffered saline solution, pH 7.4). Subsequently the two polymer solutions can be mixed and incubated at a defined temperature (e.g. 37° C.) whereby a covalently cross-linked hydrogel is formed due to the [4+2]-cyclo addition that takes place. A large number of hydrogels can be prepared using this uncomplicated process which can be adapted exactly to the requirements of certain applications.

The macromonomers are cross-linked by stepwise polymerization (or “Step Growth Polymerization”) using only Diels-Alder [4+2]-cyclo addition; free-radical polymerization reactions and/or physical cross-linking principles (e.g. electrostatic or hydrophobic interactions) are not employed. The reaction of the macromonomers is shown by way of example in the reaction equation 2 below, according to which PEG macromonomers functionalized with a furyl group as a diene react with PEG macromonomers functionalized with a maleimide group as a dienophile in a [4+2]-cyclo addition to form covalently cross-linked hydrogels. The broken bond shown at the end of the molecules opposite the diene and the dienophile illustrates that only a part of the macromonomers is shown since additional reactive groups are present for the cross-linking as described above (cf. also FIG. 1).

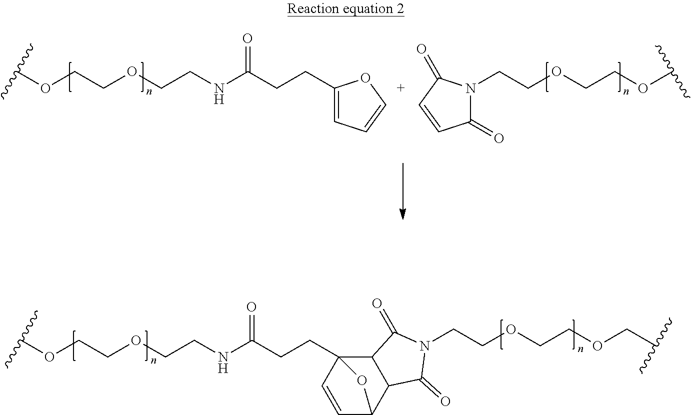

The hydrogels obtainable by Diels-Alder reaction exhibit a defined architecture, controllable mesh width and an extremely low sol fraction. The polymer content of the hydrogels is typically below 25% (w/v, based on g/ml); in the swollen state, the water content can be up to 95% (w/v). Furthermore, the hydrogels are preferably characterized by excellent optical transparency which allows a large number of biomedical (e.g. ophthalmological) and technical applications. The process described above or the resulting hydrogel, respectively, are especially suitable for providing a scaffolding material for living cells, e.g. implanted or migrated cells, or for providing a carrier system for the controlled release of drugs, such as drugs of biogenic origin, preferably peptides, proteins or nucleic acids in biologically active form (cf. FIG. 2). Preferred embodiments of the present invention are therefore (a) a hydrogel as described above obtainable by cross-linking macromonomers by means of Diels-Alder [4+2]-cyclo addition and additionally comprising living cells, and (b) a hydrogel as described above obtainable by cross-linking macromonomers by means of Diels-Alder [4+2]-cyclo addition and additionally comprising a drug. The drug can be fixed in the hydrogel exclusively by the formed network. However, it can also be present in the hydrogel in covalently bonded form. With respect to embodiment (b), a distinction can be made between two preferred scenarios b1) and b2) which are difficult to realize with conventional cross-linking reactions:

b1) The functional groups of peptides, proteins or nucleic acids (e.g. alcohols, amines, carboxylic acids, etc.) present in the hydrogel are largely inert towards the Diels-Alder cross-linking reaction; an uncontrolled and therefore activity-decreasing cross-linking of peptides, proteins or nucleic acids is effectively prevented. Drugs of biogenic origin can already be “packaged” in hydrogels during cross-linking, which guarantees an extremely high loading efficiency. At the same time, however, they can quickly be released in biologically active form.

b2) Peptides, proteins or nucleic acids can be derivatized with suitable linker molecules such that they are reversibly bonded to the gel network during the cross-linking reaction. Suitable linker molecules include, for example, a diene or dienophile functionality. Covalent bonding to the gel network secures the peptides, proteins or nucleic acids in the hydrogel. In accordance with the degradation kinetics of the linker molecules, they can for example be released to the environment in a prolonged way. This approach is especially promising if the goal is a therapy with a biogenic drug for an extended period of time.

The hydrogel according to the present invention typically exhibit good biocompatibility. As a general rule, they can be degraded in the organism after they have served their purpose. If desired, the degradation can additionally be accelerated by modifying the macromonomers. If the starting polymers used are not biodegradable to begin with (such as e.g. PEG), they can be modified for example by the incorporation of a hydrolytically cleavable sequence or an enzymatically cleavable sequence. This ensures for example that the after application in an organism, the hydrogels of the present invention can also be degraded there. This biodegradability can be advantageous for the use as scaffolding material for living cells or as carrier material for drugs. Of course, the incorporation of hydrolytically or enzymatically cleavable sequences is also suitable for additionally controlling the degradation rate of a hydrogel according to the present invention or to accelerate it if a starting polymer is used which can degrade as such in a patient's organism.

A single example of a hydrolytically cleavable sequence e.g. oligomers of lactic acid can be mentioned. Single ester groups can also control the degradation kinetics of the hydrogel as hydrolytically cleavable groups. As examples of enzymatically cleavable sequences known peptide sequences shall be mentioned which can be cleaved by matrix metalloproteases (MMP) (e.g. the Pro-Leu-Gly-Leu-Trp-Ala-Arg motif). The incorporation of a hydrolytically or enzymatically cleavable sequence can advantageously take place between the terminal group of the starting polymer and the diene or dienophile to be attached. This process is shown by way of example with the structure of formula (6) below, wherein a cleavable sequence Z was incorporated between a PEG as polymer and a functional group with a furyl group as diene.

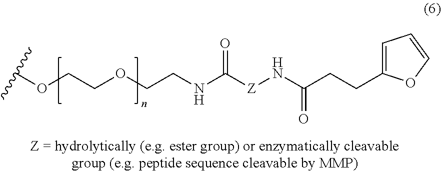

The introduction of a cleavable group poses no problem since reactions can be used for the functionalization of the starting polymers which are compatible e.g. with the standard methods of peptide synthesis. After cross-linking the macromonomers by means of Diels-Alder reaction, hydrolytically or enzymatically degradable hydrogels are formed. The use of enzymatically cleavable sequences offers the advantage that the degradation of the cross-linked hydrogels does not proceed at a consistent rate but rather depends on the biological circumstances at the application site (e.g. enzyme expression). By varying the number and type of the enzymatically cleavable sequences, the degradation rate of the hydrogels can be accelerated or delayed depending on the application. This possibility represents yet another innovation compared to known hydrogels whose degradation rate cannot be controlled at all or only with great difficulty.

In addition to cleavable sequences, other molecules or substances, such as e.g. peptide sequences promoting cell adhesion (e.g. the Arg-Gly-Asp-Sermotif), cyclodextrins, dyes for detection or nanoparticles, can be bonded directly to the gel scaffold. For this purpose, these molecules have be functionalized either with a diene or a dienophile. This high degree of flexibility in terms of the design of the hydrogels opens up numerous possible applications in the field of tissue engineering. By incorporating peptide sequences specifically promoting cell adhesion, such as e.g. the Arg-Gly-Asp-Ser or the Tyr-Ile-Gly-Ser-Arg motif, the adhesion, proliferation, migration and differentiation of implanted or migrated cells can be specifically influenced. Moreover, due to the defined architecture of the macromonomers used in the process and the high efficiency of the Diels-Alder cross-linking reaction, mesh width, swelling degree and mechanical properties of the hydrogels can be controlled. This is also of significant importance in tissue engineering since the physicochemical properties of the cell carrier also influence adhesion, proliferation, migration and differentiation of implanted or migrated cells.

Generally, the hydrogels of the present invention can comprise further additives. These can also be molecular components for detection, marking or active interaction with the environment.

As was explained above, the described hydrogels can be used for numerous applications. Examples of suitable applications include (a) a carrier system for a drug, in particular a biogenic drug, such as a peptide, a protein or a nucleic acid; (b) a scaffolding material for living cells, in particular cells implanted or migrated into the scaffolding material; (c) hydrogels for ophthalmological applications, i.e. in particular for the treatment of diseases affecting the eye; (d) a filler material, in particular for biomedical applications wherein the hydrogel as such is typically applied to the a patient. These possible applications are of particular importance for the treatment, especially the therapeutic treatment, of humans or animals.

Due to the reaction mechanism employed in the preparation of the hydrogels, the gels according to the present invention are capable of gelling in situ, i.e. at the application site in a living organism of a patient to be treated (human or animal). The use of excipients, e.g. catalysts, or activation energy is usually not necessary. The macromonomers can therefore conveniently be applied, e.g. injected, in the form of a polymer solution to then solidify into a gel at the application site. Examples of polymer solutions include a solution containing a mixture of the macromonomers, or two separate solutions containing the diene macromonomers and the dienophile macromonomers, respectively. Preferred polymer contents of polymer solutions especially suitable for in situ gelling are in the range of 1 to 30%, in particular 2.5 to 20% (w/v, based on g/ml).

Thus, for applying the hydrogel both components that make up the gel, i.e. the diene macromonomers and the dienophile macromonomers, can be mixed immediately prior to the introduction into a human or animal organism, for example prior to an injection. The mixture can preferably be provided in the form of an aqueous solution. Other desired components, such as a drug and/or conventional pharmaceutical excipients, can also be added to the mixture. Alternatively, the components can be injected using a dual chamber syringe, optionally together with a drug and/or one or more excipients. Here as well, it is advantageous to provide the solutions to be injected as aqueous solutions. Finally, it is of course also possible to apply the components one after the other, for example by way of quick successive injections at the same application site.

The cross-linking of the macromonomers then takes place in situ, i.e. directly at the application site in the living organism. This way, a high degree of variability of the external shape of the hydrogels is ensured; the external shape of the hydrogels adapts to the anatomical situation at the application site. The hydrogels according to the present invention often form three-dimensional structures with a volume in the microliter to milliliter range, e.g. 1 μl to 10 ml. However, they are also suitable for coating surfaces wherein due to the desired cross-linking coating thicknesses of at least 500 nm to 1 mm, in particular 1 μm to 1 mm are typically preferred. The application by means of minimally invasive methods is especially gentle for the patient since no large skin incisions are required. Furthermore, this allows the application of the hydrogels in tissue which is not readily accessible anatomically or in body cavities. Examples include the interior of the eye, the inner ear, the frontal sinus cavity, the sinuses, and the dental pulp. The sterility of the hydrogels can be ensured by sterile filtration of the polymeric starting solutions.

Thus, the present invention is also directed to a process for the controlled administration of a drug to a patient to be treated, comprising the application of the one of the hydrogels described above, or of the macromonomers necessary to form the hydrogel, together with the drug to a patient.

The present invention is also directed to a process for building tissue within the framework of tissue engineering, comprising the application of the one of the hydrogels described above, or of the macromonomers necessary to form the hydrogel, together with the living cells necessary to build tissue to a patient.

Preferred embodiments of the invention are additionally summarized in the following points:
1. Hydrogel prepared by covalent cross-linking of macromonomers by means of Diels-Alder [4+2] cyclo addition.
2. Hydrogel according to point 1, wherein at least one macromonomer with at least two diene functions and at least one macromonomer with at least two dienophile functions are used in the preparation.
3. Hydrogel according to point 1 or 2, formed from hydrophilic star or comb polymers of synthetic origin, in particular star or comb polymers with a molecular weight of 2-20 kDa.
4. Hydrogel according to one of the preceding points, consisting of polyethylene glycol (PEG).
5. Hydrogel according to one of the preceding points which is immediately cross-linked at the application site after administration of the liquid precursors.
6. Hydrogel according to one of the preceding points which is biodegradable due to the incorporation of hydrolytically or enzymatically cleavable sequences.
7. Hydrogel according to one of the preceding points comprising additional molecular components for detection, marking or active interaction with the environment.
8. Hydrogel according to one of the preceding points comprising biogenic drugs (e.g. peptides, proteins or nucleic acids).
9. Hydrogel according to point 8 comprising biogenic drugs (e.g. peptides, proteins or nucleic acids) in covalently bonded form.
10. Hydrogel according to one of the preceding points comprising living cells.
11. Hydrogel according to one of the preceding points which is used a filler material for biomedical applications.
12. Hydrogel according to one of the preceding points which is used as a carrier system in particular for the controlled release of drugs.
13. Hydrogel according to one of the preceding points which is used as scaffolding material for implanted or migrated cells.
14. Hydrogel according to one of the preceding points for use in ophthalmological applications.
15. Use of a hydrogel according to one of points 1 to 13 in the treatment of diseases affecting the eye.

### EXAMPLES

### Preparation Example 1

### Functionalization of Star-Shaped PEG with Furyl Groups

The synthesis is described by way of example for a star-shaped PEG (degree of branching four, molecular weight 10 kDa, 4armPEG10 k-OH). For preparing the starting material, 4armPEG10 k-OH was first converted into the corresponding amine (4armPEG 10 k-NH₂) as described in the literature (e.g. Brandl, F., Henke, M., Rothschenk, S., Gschwind, R., Breunig, M., Blunk, T., Tessmar, J. and Göpferich, A. (2007), Poly(Ethylene Glycol) based Hydrogels for Intraocular Applications. Adv. Eng. Mater., 9: 1141-1149). Then 224 mg (1.6 mmol) 3-(2-furyl)propionic acid, 184 mg (1.6 mmol) N-hydroxysuccinimide and 330 mg (1.6 mmol) N,N′-dicyclohexylcarbodiimide were dissolved in 10 ml 1,4-dioxane (Solution A). The reaction mixture was stirred for 6 hours at room temperature. In a separate container, 2 g (0.2 mmol) 4armPEG10 k-NH₂ and 134 mg (1.6 mmol) sodium hydrogencarbonate were dissolved in 10 ml water (Solution B). Solution A was filtered, added to Solution B, and stirred overnight at 50° C. The next day, the solvent was removed and the residue was dissolved in 20 ml water. The raw product was extracted four times with 20 ml dichloromethane each. The combined organic phases were dried over anhydrous sodium sulfate and evaporated to about 5 ml using a rotary evaporator. At 0° C., the product was precipitated by the dropwise addition of 50 ml diethyl ether. The resulting precipitate was collected by means of filtration, washed with cold diethyl ether and dried under vacuum (yield: 80%).

¹H-NMR (CDCl₃, 300 MHz): δ 2.50 ppm (t, 8H, —NHC(O)CH₂CH₂—), 2.97 ppm (t, 8H, —NHC(O)CH₂CH₂—), 3.39 ppm (s, 8H, RCCH₂O—), 3.62 ppm (s, —OCH₂CH₂—), 6.00 ppm (s, 4H, Ar), 6.25 ppm (s, 4H, Ar), 7.27 ppm (s, 4H, Ar)

### Preparation Example 2

### Functionalization of Star-Shaped PEG with ETPI

The synthesis is described by way of example for a star-shaped PEG (degree of branching four, molecular weight 10 kDa, 4armPEG10 k-OH). In the first step, 2 g (0.2 mmol) 4armPEG10 k-OH were dissolved in 25 ml toluene and dried by means of azeotropic distillation. Then the remaining solvent was removed with the rotary evaporator. The residue was dissolved in 15 ml dichloromethane together with 393 mg (2.4 mmol) 3,6-epoxy-1,2,3,6-tetrahydrophthalimide (ETPI) and 314 mg (1.2 mmol) triphenylphosphine. The mixture was cooled to 0° C. in an ice bath; subsequently, 240 μl diisopropylazodicarboxylate (dissolved in 2.5 ml dichloromethane) were added drop by drop. The reaction mixture was stirred at room temperature for 48 hours; then the solvent was removed with the rotary evaporator. For purification, the raw product was dissolved in 50 ml water and washed twice with 50 ml diethyl ether each time. The aqueous phase was completely removed with the rotary evaporator. The raw product was dissolved in 5 ml dichloromethane and precipitated at 0° C. by the dropwise addition of 50 ml diethyl ether. The resulting precipitate was collected by means of filtration, washed with cold diethyl ether and dried under vacuum (yield: 95%).

¹H-NMR (CDCl₃, 300 MHz): δ 2.84 ppm (s, 4H, exo) 3.39 ppm (s, 8H, RCCH₂O—), 3.62 ppm (s, —OCH₂CH₂—), 5.24 ppm (s, 4H, endo), 5.29 ppm (s, 4H, exo), 6.40 ppm (s, 4H, —CH═CH—, endo), 6.50 ppm (s, 4H, —CH═CH—, exo)

### Preparation Example 3

### Functionalization of Star-Shaped PEG with Maleimide Groups

The synthesis is described by way of example for a star-shaped PEG (degree of branching four, molecular weight 10 kDa, 4armPEG10 k-OH). 4armPEG10 k-OH was first functionalized with ETPI as described in Preparation Example 2. Then 2 g of the reaction product of Preparation Example 2 was dissolved in 25 ml toluene. This solution was refluxed for 2 hours at 120° C. using argon as a protective gas. After that, the solvent was removed with the rotary evaporator. The raw product was dissolved in 5 ml dichloromethane and precipitated at 0° C. by the dropwise addition of 50 ml diethyl ether. The resulting precipitate was collected by means of filtration, washed with cold diethyl ether and dried under vacuum (yield: 85%).

¹H-NMR (CDCl₃, 300 MHz): δ 3.39 ppm (s, 8H, RCCH₂O—), 3.62 ppm (s, —OCH₂CH₂—), 6.69 ppm (s, 8H, —C(O)CH═CHC(O)—)

### Example 1

### Preparation of Hydrogels by Means of Diels-Alder Reaction

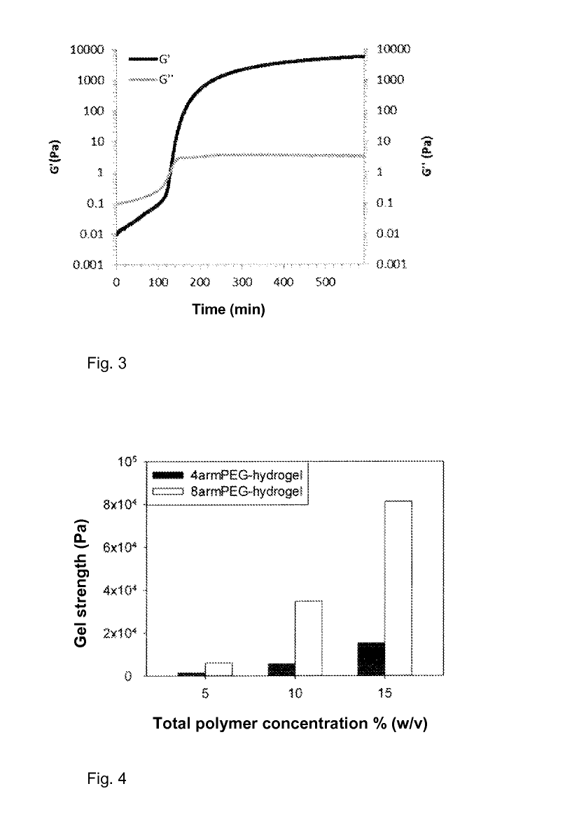

The preparation of the gel is described by way of example for macromonomers of star-shaped PEG (degree of branching four, molecular weight 10 kDa, 4armPEG10 k-OH); the total polymer content is 10% (w/v, here and hereinafter based on g/ml, unless defined otherwise). For the preparation of the hydrogels, macromonomers functionalized with furyl groups of Preparation Example 1 and macromonomers functionalized with maleimide groups of Preparation Example 3 were used. 50 mg of each of the two macromonomers were dissolved in 500 μl water each. Then the two polymer solutions were combined and incubated at 37° C. Due to the [4+2]-cyclo addition which took place, the macromonomers were cross-linked to form elastic hydrogels. FIG. 3 shows the rheogram of one of the hydrogels with 10% (w/v) total polymer content. The measurement was carried out using an oscillating rheometer (AR 2000 from TA Instruments, 40 mm plate as measurement geometry) at 37° C.

### Preparation Example 4

### Functionalization of Star-Shaped PEG with Maleimide Groups

The functionalization of star-shaped PEG with a degree of branching of four (molecular weight 10 kDa, 4armPEG10 k-OH) was described in Preparation Example 3. A different method was used to functionalize star-shaped PEG with a degree of branching of eight (molecular weight 10 kDa, 8armPEG10 k-OH). First, the starting material was converted into the corresponding amine (8armPEG10 k-NH₂) as described in the literature (e.g. Brandl, F., Henke, M., Rothschenk, S., Gschwind, R., Breunig, M., Blunk, T., Tessmar, J. and Göpferich, A. (2007), Poly(Ethylene Glycol) Based Hydrogels for Intraocular Applications. Adv. Eng. Mater., 9: 1141-1149). Then, 2.46 mg 8armPEG 10 k-NH₂ (0.25 mmol) were dissolved in 100 ml saturated sodium bicarbonate solution. The solution was cooled to 0° C. in an ice bath and 620 mg (4 mmol) N-methoxycarbonylmaleimide were added. After 30 min of stirring at 0° C., the ice bath was removed and the solution was slowly heated to room temperature. Then the raw material was extracted three times with 115 ml dichloromethane each. The combined organic phases were dried over anhydrous sodium sulfate and evaporated to about 6 ml using the rotary evaporator. The product was precipitated at 0° C. by the dropwise addition of 60 ml diethyl ether. The resulting precipitate was collected by means of filtration, washed with cold diethyl ether and dried under vacuum (yield: 73%).

¹H-NMR (CDCl₃, 300 MHz): δ 3.39 ppm (s, 16H, RCCH₂O—), 3.62 ppm (s, —OCH₂CH₂—), 6.69 ppm (s, 16H, —C(O)CH═CHC(O)—)

### Rheological Characterization of Hydrogels

The hydrogels were prepared using the method described in Example 1. The 4armPEG-hydrogel was prepared from macromonomers functionalized with furyl groups of Preparation Example 1 and macromonomers functionalized with maleimide groups of Preparation Example 3. The 8armPEG-hydrogel was prepared from macromonomers functionalized with furyl groups, prepared according to the method of Preparation Example 1 with the exception that 8armPEG10 k-OH was used, and macromonomers functionalized with maleimide groups of Preparation Example 4.

Gelling time and gel strength were measured using an oscillating rheometer (AR 2000 from TA Instruments, 40 mm plate as measurement geometry) at 37° C. The gel properties were essentially determined by the total polymer content and the degree of branching of the macromonomers. As the total polymer content and the degree of branching increased, the gelling time decreased (FIG. 5). For instance, the gelling time decreased from 206 min (5% 4armPEG-hydrogel) to 13 min (15% 8armPEG-hydrogel). Conversely, the gel strength significantly increased as the total polymer content and the degree of branching increased (FIG. 4). By increasing the total polymer content and the degree of branching of the macromonomers used in the process, it was possible to increase the gel strength from about 1 kPa (5% 4armPEG-hydrogel) to about 80 kPa (15% 8armPEG-hydrogel).

### Swelling and Degradation Behavior of Hydrogels

Hydrogels were prepared using the process described in Example 1 to examine their swelling and degradation behavior. The 4armPEG-hydrogel was prepared from macromonomers functionalized with furyl groups of Preparation Example 1 and macromonomers functionalized with maleimide groups of Preparation Example 3. The 8armPEG-Hydrogel was prepared from macromonomers functionalized with furyl groups, prepared according to the method of Preparation Example 1 with the exception that 8armPEG10 k-OH was used, and macromonomers functionalized with maleimide groups of Preparation Example 4.

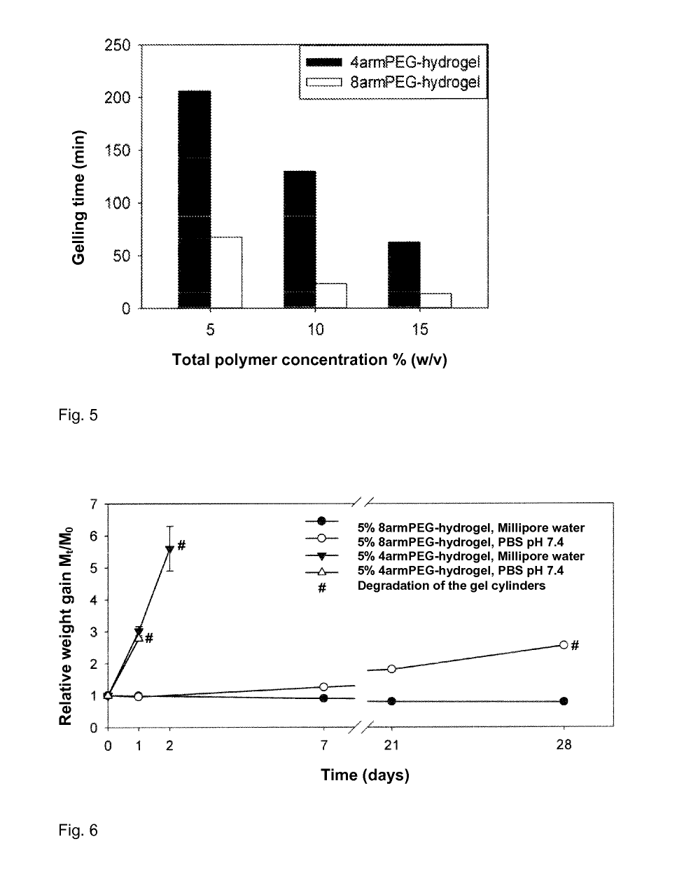

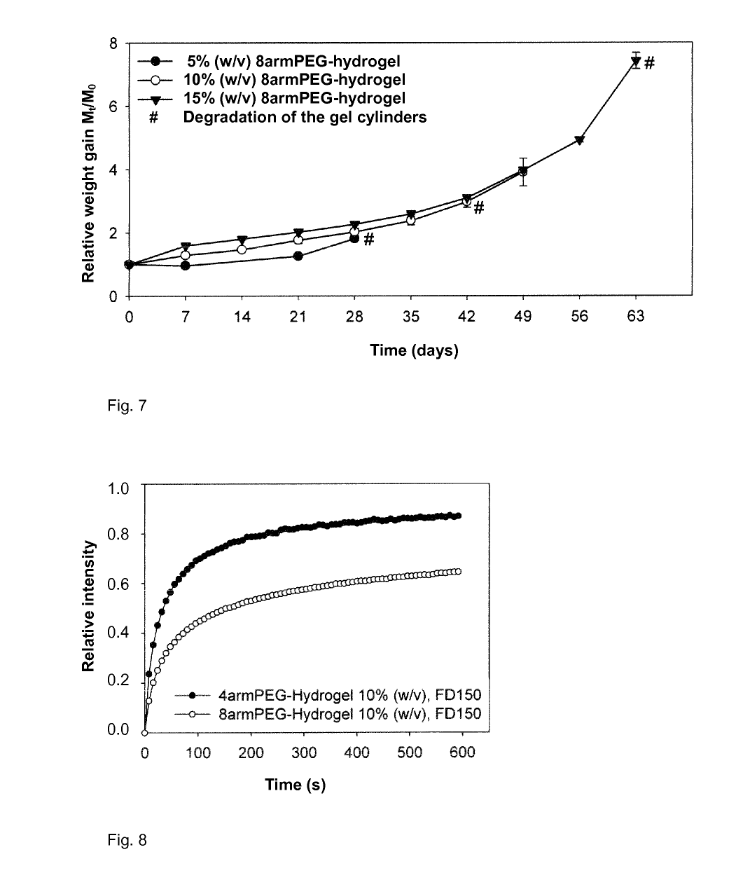

In order to study the swelling behavior, the liquid precursor mixtures were poured into glass cylinders where they gelled for 72 hours. After that, the samples were taken and incubated at 37° C. in different media (water and phosphate buffer pH 7.4). The wet weight of the hydrogels was determined at regular intervals and compared to the original weight. 4armPEG-hydrogels swell significantly more than 8armPEG-hydrogels; degradation took place after one (phosphate buffer pH 7.4) and two days (water), respectively. By increasing the degree of branching of the macromonomers, it was possible to significantly increase the stability of the hydrogels (FIG. 6). 8armPEG-hydrogels were stable for 28 days in phosphate buffer pH 7.4; neither swelling nor degradation could be observed in water during the testing period. The examination of the swelling and degradation behavior also showed a strong dependency on the total polymer concentration (FIG. 7). 8armPEG-hydrogels with 5% (w/v), 10% (w/v) and 15% (w/v) total polymer content were stable in phosphate buffer pH 7.4 for 28, 42 and 63 days, respectively.

Diffusion Von Active Ingredients in and Release from Hydrogels

### General Description

FRAP experiments (“Fluorescence recovery after photobleaching”) were carried out to examine the diffusion behavior of active ingredients in the hydrogels of the present invention, which also allow predictions regarding the release of the hydrogels.

The method is suitable for determining the diffusion rate of a molecule in the hydrogel matrix. Fluorescently-labeled dextran (average molecular weight 150 kDa, hereinafter also referred to as “FD150”) was incorporated into the Diels-Alder hydrogel as a model molecule. The sample was observed under a confocal microscope. First, the fluorescence intensity in a defined area was determined. Then, this area was bleached by means of a short laser pulse. This causes the fluorescent molecules to irreversibly change into a non-fluorescent state. If the molecules in the system are mobile, unbleached and still fluorescent model molecules can diffuse into the bleached area from the surrounding areas. By measuring the time lapsed until the bleached area is once again fluorescent, it is possible to determine the diffusion constant.

### Process Steps

Preparation of the test systems: Fluorescently-labeled dextran with an average molecular weight of 150 kDa (FD150) was used as a model molecule. The final concentration of the molecule was 1 mg/mL. 4arm- and 8armPEG10 k-hydrogels with a total polymer concentration of 10% (w/v) each were used as test systems. The amounts of the gel components needed for the preparation of the gels were dissolved in appropriate amounts of water, FD150 was added, the components were mixed and gelled overnight at room temperature.

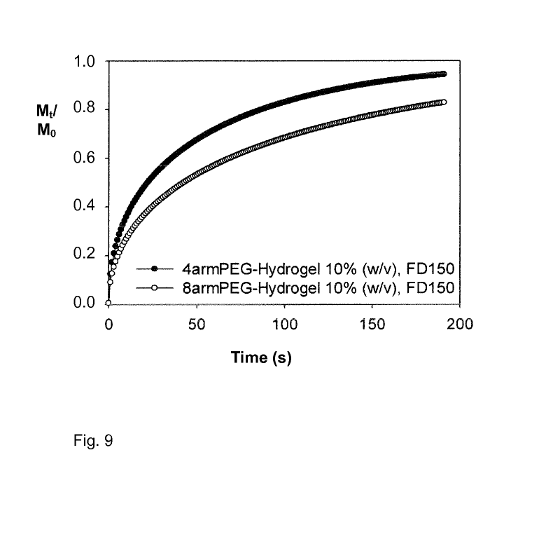

The FRAP experiments were carried out with a confocal microscope (Zeiss LSM 510, 10× lens). All the experiments were carried out with the 488 nm line of a 30 mW Ar-ion laser at an output performance of 25%. The confocal aperture was opened to its maximum setting so that as much fluorescence as possible could be detected. Whenever an area in the test system was focused, a time series of digital images was recorded at a low laser intensity (0.2% transmission). The time interval between two successive images was 1.8 s. After five images of the unbleached state, circular areas with a diameter of 36 μm each were bleached at maximum laser intensity (100% transmission). The duration of the bleaching process was selected to be as short as possible in order to prevent that fluorescence returns while the bleaching is still ongoing. After bleaching, a series of 75 images was recorded once again at low laser intensity (0.2% transmission) to observe the recovery of the fluorescence in the bleached area in relation to the time (FIG. 8). Then the recorded images were evaluated using the software Image J (U.S. National Institutes of Health) and the chronological sequence of the fluorescence intensity in the bleached area was determined. By adapting suitable systems of equations to the experimental data, the diffusion coefficient D for the model molecule FD150 was determined in the different systems (cf. Braeckmans, K. et al.; Biophys. J., 2003, 85, 2240-2252). With the help of the calculated diffusion coefficient D and another model based on Fick's law, predictions were made with respect to the release behavior of the molecules in the different systems (cf. Siepmann, J.; Siepmann, F.; Int. J. Pharm., 2008, 364, 328-343) (FIG. 9).

1. A method of making a hydrogel comprising the step of covalent cross-linking of macromonomers by means of Diels-Alder [4+2] cyclo addition.

2. The method according to claim 1, further comprising the step of forming the hydrogel from star or comb polymers of synthetic origin.

3. The method according to claim 1, wherein the macromonomers have a molecular weight of 2-20 kDa.

4. The method according to claim 1, further comprising the step of using polyethylene glycol as polymeric starting material for the macromonomers.

5. The method according to claim 1, wherein hydrolytically or enzymatically cleavable sequences are incorporated into the hydrogel structure.

6. The method according to claim 1, further comprising additional molecular components for detection, marking or active interaction with the environment.

7. The method according to claim 1, further comprising a drug.

8. The method according to claim 7, wherein the drug is contained in covalently bonded form.

9. The method according claim 7, wherein the drug is a peptide, a protein or a nucleic acid.

10. The method according to claim 1, comprising living cells.

11. The method according to claim 1 for use in a process for the therapeutic treatment of the human or animal body, wherein the treatment comprises the in situ cross-linking of the hydrogel at the application site in a living organism.

12. The method according to claim 1 for use as a filler material for biomedical applications.

13. The method according to claim 1 for use as a carrier system for the controlled release of drugs.

14. The method according to claim 1 for use as a scaffolding material for implanted or migrated cells.

15. The method according to claim 1 for use in the treatment of diseases affecting the eye.


SEQUENCE LISTING (3 sequences)

SEQ ID NO: 1 - protein, 7 aa, Artificial sequence
  /note=“Description of artificial sequence MMP cleavable sequence”
PLGLWAR

SEQ ID NO: 2 - protein, 4 aa, Artificial sequence
  /note=“Description of artificial sequence sequence 1 for promoting cell adhesion”
RGDS

SEQ ID NO: 3 - protein, 5 aa, Artificial sequence
  /note=“Description of artificial sequence sequence 2 for promoting cell adhesion”
YIGSR